(19) United States
(12) Patent Application Publication
(10) Pub. No.: US 2006/0224226 A1
(43) Pub. Date: Oct. 5, 2006

(54) In-vivo radial orientation of a polymeric implantable medical device

(76) Inventors: Bin Huang, Pleasanton, CA (US); Klaus Kleine, Los gatos, CA (US); David C. Gale, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/096,342

(22) Filed: Mar. 31, 2005

(51) Int. Cl. A61F 2/06

(52) U.S. Cl. 623/1.11

(57) ABSTRACT

A method and system for treating a bodily lumen with an implantable medical device, such as a stent, are disclosed. The device may be disposed within a bodily lumen and radially expanded by circumferentially deforming a tube-like section of the device. The deforming section may expand the lumen and the deformed section may support the lumen.

### BACKGROUND OF THE INVENTION

1. Field of the Invention

This invention relates to radial deformation of an implantable medical device, such as a stent, in vivo after implantation of the device in a bodily lumen.

2. Description of the State of the Art

This invention relates to radially expandable endoprostheses, which are adapted to be implanted in a bodily lumen. An “endoprosthesis” corresponds to an artificial device that is placed inside the body. A “lumen” refers to a cavity of a tubular organ such as a blood vessel.

A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen. In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a retractable sheath or a sock. When the stent is in a desired bodily location, the sheath may be withdrawn which allows the stent to self-expand.

The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may be also be described as, hoop or circumferential strength and rigidity.

Additionally, the stent should also be longitudinally flexible to allow it to be maneuvered through a tortuous vascular path and to enable it to conform to a deployment site that may not be linear or may be subject to flexure. The material from which the stent is constructed must allow the stent to undergo expansion. Once expanded, the stent must maintain its size and shape throughout its service life despite the various forces that may come to bear on it, including the cyclic loading induced by the beating heart. Finally, the stent must be biocompatible so as not to trigger any adverse vascular responses.

The structure of a stent is typically composed of scaffolding that includes a pattern or network of interconnecting structural elements or struts. The scaffolding can be formed from wires, tubes, or sheets of material rolled into a cylindrical shape. The scaffolding is designed so that the stent can be radially contracted (to allow crimping) and radially expanded (to allow deployment). A conventional stent is allowed to expand and contract through movement of individual structural elements of a pattern with respect to each other. Such movement typically results in substantial deformation of localized portions of the stent's structure.

The pattern should be designed to maintain the longitudinal flexibility and radial rigidity required of the stent. Longitudinal flexibility facilitates delivery of the stent and radial rigidity is needed to hold open a bodily lumen.

Stents have been made of many materials such as metals and polymers, including biodegradable polymer materials. A medicated stent may be fabricated by coating the surface of either a metallic or polymeric scaffolding with a polymeric carrier that includes an active agent or drug. In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodable materials such as bioabsorbable polymers may be configured to meet this additional clinical requirement since they may be designed to completely erode after the clinical need for them has ended.

Conventional methods of constructing a stent from a polymer material involve extrusion of a polymer tube based on a single polymer or polymer blend and then laser cutting a pattern into the tube. An advantage of stents fabricated from polymers is that they can possess greater flexibility than metal stents. Other potential shortcomings of metal stents include adverse reactions from the body, nonbioerodability, and non-optimal drug-delivery.

A disadvantage of polymer stents compared to metal stents, is that polymer stents typically have less circumferential strength and radial rigidity. Inadequate circumferential strength potentially contributes to a relatively high incidence of recoil of polymeric stents after implantation into vessels. The requirement of high strength and rigidity is seemingly at odds with the need for flexibility during delivery. However, the movable structural elements in the stent pattern do provide some flexibility.

Another potential problem with polymeric stents is that their struts can crack during crimping and expansion, especially for brittle polymers. The localized portions of the stent pattern subjected to substantial deformation tend to be the most vulnerable to failure. Furthermore, in order to have adequate mechanical strength, polymeric stents may require significantly thicker struts than a metallic stent, which results in an undesirably larger profile.

Another potential problem with polymeric stents is long term creep. Long term creep is typically not an issue with metallic stents. Creep is a consequence of the viscoelastic nature of polymeric materials. Long term creep refers to the gradual deformation that occurs in a polymeric material subjected to an applied load. Long term creep occurs even when the applied load is constant.

Long term creep in a polymeric stent reduces the effectiveness of a stent in maintaining a desired vascular patency. In particular, long term creep allows inward radial forces to permanently deform a stent radially inward.

Therefore, it would be desirable to have method of treating a bodily lumen with a polymeric stent in which the stent has adequate flexibility during delivery, high radial strength and rigidity after deployment, high creep resistance after deployment, and that is relatively free of localized regions of high deformation susceptible to failure.

### SUMMARY OF THE INVENTION

The present invention is directed to embodiments of a method of treating a bodily lumen with an implantable medical device, such as a stent. The method may include disposing an implantable medical device within a bodily lumen. The device may include a tube-like section having an abluminal face and a luminal face extending between a proximal end and a distal end of the section. The method may further include radially expanding the device about a cylindrical axis of the section within the lumen by circumferentially deforming the section. The deforming section may expand the lumen and the deformed section may support the lumen.

The present invention is also directed to embodiments of a method of fabricating a system for treating a bodily lumen. The method may include disposing an implantable medical device over a delivery implement. The device may include a tube-like section having an abluminal face and a luminal face extending between a proximal end and a distal end. The delivery implement may be configured to radially expand the device within the lumen by circumferentially deforming the section about a cylindrical axis of the section. The deforming section may expand the lumen and the deformed section may support the lumen.

In another aspect of the invention, a system for treating a bodily lumen may include an implantable medical device having a tube-like section. The device may include a tube-like section having an abluminal face and a luminal face extending between a proximal end and a distal end of the section. A delivery implement may be configured to radially expand the device by circumferentially deforming the section about a cylindrical axis the section. The deforming section may expand the lumen and the deformed section may support the lumen.

### BRIEF DESCRIPTION OF THE DRAWINGS

FIG. 1 illustrates a conventional stent.

FIGS. 2A-C depict stent elements from a conventional stent.

FIG. 3 depicts a tube-like section.

FIGS. 4A-B depict a stent with tube-like sections.

FIG. 4C depicts a stent that is a tube.

FIG. 5A-B depicts a stent with tube-like sections mounted on a catheter balloon.

FIG. 6 depicts a scanning electron microscope image of a stent with tube-like sections before radial expansion.

FIGS. 7-8 depict scanning electron microscope images of a stent with tube-like sections after radial expansion.

FIG. 9 depicts a graph of results from long term creep experiments.

FIG. 10 depicts a graph of differential scanning calorimetry results.

### DETAILED DESCRIPTION OF THE INVENTION

For the purposes of the present invention, the following terms and definitions apply:

The “glass transition temperature,” T_{g}, is the temperature at which the amorphous domains of a polymer change from a brittle vitreous state to a solid deformable or ductile state at atmospheric pressure. In other words, the T_{g} corresponds to the temperature where the onset of segmental motion in the chains of the polymer occurs. When an amorphous or semicrystalline polymer is exposed to an increasing temperature, the coefficient of expansion and the heat capacity of the polymer both increase as the temperature is raised, indicating increased molecular motion. As the temperature is raised the actual molecular volume in the sample remains constant, and so a higher coefficient of expansion points to an increase in free volume associated with the system and therefore increased freedom for the molecules to move. The increasing heat capacity corresponds to an increase in heat dissipation through movement. T_{g} of a given polymer can be dependent on the heating rate and can be influenced by the thermal history of the polymer. Furthermore, the chemical structure of the polymer heavily influences the glass transition by affecting mobility.

“Stress” refers to force per unit area, as in the force acting through a small area within a plane. Stress can be divided into components, normal and parallel to the plane, called normal stress and shear stress, respectively. Tensile stress, for example, is a normal component of stress applied that leads to expansion (increase in length). In addition, compressive stress is a normal component of stress applied to materials resulting in their compaction (decrease in length). Stress may result in deformation of a material, which refers to change in length. “Expansion” or “compression” may be defined as the increase or decrease in length of a sample of material when the sample is subjected to stress.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

Furthermore, a property of a material that quantifies a degree of strain with applied stress is the modulus. “Modulus” may be defined as the ratio of a component of stress or force per unit area applied to a material divided by the strain along an axis of applied force that results from the applied force. For example, a material has both a tensile and a compressive modulus. A material with a relatively high modulus tends to be stiff or rigid. Conversely, a material with a relatively low modulus tends to be flexible. The modulus of a material depends on the molecular composition and structure, temperature of the material, and the strain rate or rate of deformation. For example, below its T_{g}, a polymer tends to be brittle with a high modulus. As the temperature of a polymer is increased from below to above its T_{g}, its modulus decreases.

The “ultimate strength” or “strength” of a material refers to the maximum stress that a material will withstand prior to fracture. A material may have both a tensile and a compressive strength. The ultimate strength may be calculated from the maximum load applied during a test divided by the original cross-sectional area.

The term “elastic deformation” refers to deformation of an object in which the applied stress is small enough so that the object moves towards its original dimensions or essentially its original dimensions once the stress is released. However, an elastically deformed polymer material may be prevented from returning to an undeformed state if the material is below the T_{g} of the polymer. Below T_{g}, energy barriers may inhibit or prevent molecular movement that allows deformation or bulk relaxation.

“Elastic limit” refers to the maximum stress that a material will withstand without permanent deformation. The “yield point” is the stress at the elastic limit and the “ultimate strain” is the strain at the elastic limit. The term “plastic deformation” refers to permanent deformation that occurs in a material under stress after elastic limits have been exceeded.

Brittle materials are relatively stiff or rigid materials that exhibit little or no plastic deformation. As the stress applied to a brittle material increases, it tends to fracture at a stress approximately equal to its ultimate strength, undergoing little or no plastic deformation. For example, a polymer below its T_{g} tends to be brittle. On the other hand, a ductile material under an applied stress exhibits both elastic and plastic deformation prior to fracture. Above its T_{g}, a polymer is ductile.

“Creep” refers to the increase in strain with time in a polymeric material under a constant load.

A stent made from a biodegradable polymer is intended to remain in the body for a duration of time until its intended function of, for example, maintaining vascular patency and/or drug delivery is accomplished. After the process of degradation, erosion, absorption, and/or resorption has been completed, no portion of the biodegradable stent, or a biodegradable portion of the stent will remain. In some embodiments, very negligible traces or residue may be left behind. The duration is typically in the range of six to eighteen months.

In general, polymers can be biostable, bioabsorbable, biodegradable, or bioerodable. Biostable refers to polymers that are not biodegradable. The terms biodegradable, bioabsorbable, and bioerodable, as well as degraded, eroded, and absorbed, are used interchangeably and refer to polymers that are capable of being completely eroded or absorbed when exposed to bodily fluids such as blood and can be gradually resorbed, absorbed and/or eliminated by the body.

Representative examples of polymers that may be used to fabricate embodiments of implantable medical devices disclosed herein include, but are not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers that may be especially well suited for use in fabricating embodiments of implantable medical devices disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, poly(vinyl acetate), styrene-isobutylene-styrene triblock copolymers, and polyethylene glycol.

The chemical and mechanical properties of a polymer are largely dependent on the microstructure or molecular arrangement of component polymer molecules. An amorphous microstructure of a solid state polymer is characterized by a random or disordered state. An amorphous polymer tends not to have regions in which polymer chains are aligned along an axis with respect to one another.

Alternatively, solid state polymers may also have regions with an oriented microstructure in which polymer chains are disordered, but are aligned or oriented along a longitudinal or covalent axis. Furthermore, crystalline regions are characterized by polymer chains that are ordered so that the polymer chains fit together in a lattice. Finally, a polymer may have regions that are both oriented and crystalline. In an oriented crystalline region, polymer chains fit together in an ordered lattice and are aligned along an axis.

Oriented crystalline regions tend to have high strength and high modulus (low elongation with applied stress) along an axis of alignment of the polymer chains. Additionally, oriented crystalline regions tend to have a higher T_{g} than amorphous regions.

Additionally, orientation and crystallinity may be induced in a polymer by applying stress to induce strain in the polymer. This process is referred to as strain-induced crystallization. Therefore, chemical and mechanical properties such as strength, modulus, and T_{g} may be modified by inducing molecular orientation and crystallinity in a polymer.

The degree of polymer chain alignment induced by strain may depend upon the temperature of the polymer. For example, below the glass transition temperature, T_{g}, of a polymer, polymer segments may not have sufficient energy to move past one another. In general, polymer chain alignment may not be induced without sufficient segmental mobility.

Above T_{g}, polymer chain alignment may be readily induced with applied stress since rotation of polymer chains, and hence segmental mobility occur relatively easily. Between T_{g} and the melting temperature of the polymer, Tₘ, rotational barriers exist. However, the barriers are not great enough to substantially prevent segmental mobility. As the temperature of a polymer is increased above T_{g}, the energy barriers to rotation decrease and segmental mobility of polymer chains tends to increase. As a result, as the temperature increases, polymer chain alignment is more easily induced with applied stress. Therefore, heating a polymer may facilitate strain-induced crystallization.

Rearrangement of polymer chains may take place when a polymer is stressed in an elastic region and in a plastic region of the polymer material. A polymer stressed beyond its elastic limit to a plastic region generally retains its stressed configuration and corresponding induced polymer chain alignment when stress is removed. The polymer chains may become oriented in the direction of the applied stress which results in an oriented crystalline structure. Thus, applying strain-induced crystallization to a polymer sample may result in a permanently deformed high strength, high modulus material with a higher T_{g} than the original sample.

The term “implantable medical device” is intended to include, but is not limited to, balloon-expandable stents, stent-grafts, and vascular grafts. In general, implantable medical devices, such as stents, can have virtually any structural pattern that is compatible with a bodily lumen in which it is implanted. Typically, a stent is composed of a pattern or network of circumferential and longitudinally extending interconnecting structural elements or struts. In general, the struts are arranged in patterns, which are designed to contact the lumen walls of a vessel and to maintain vascular patency. A myriad of strut patterns are known in the art for achieving particular design goals. A few of the more important design characteristics of stents are radial or hoop strength, expansion ratio or coverage area, and longitudinal flexibility.

Polymer tubes used for fabricating stents may be formed by various methods. These include, but are not limited to extrusion or injection molding. Conventionally extruded tubes tend to possess no or substantially no radial orientation or, equivalently, polymer chain alignment in the circumferential direction.

A tube used for fabricating a stent may be cylindrical or substantially cylindrical in shape. In some embodiments, the diameter of the polymer tube prior to fabrication of an implantable medical device may be between about 0.2 mm and about 5.0 mm, or more narrowly between about 1 mm and about 3 mm.

In general, the scaffolding of conventional stents is designed so that the stent can be radially contracted (to allow crimping) and radially expanded (to allow deployment). A conventional stent is allowed to expand and contract through movement of individual structural elements of a pattern with respect to each other.

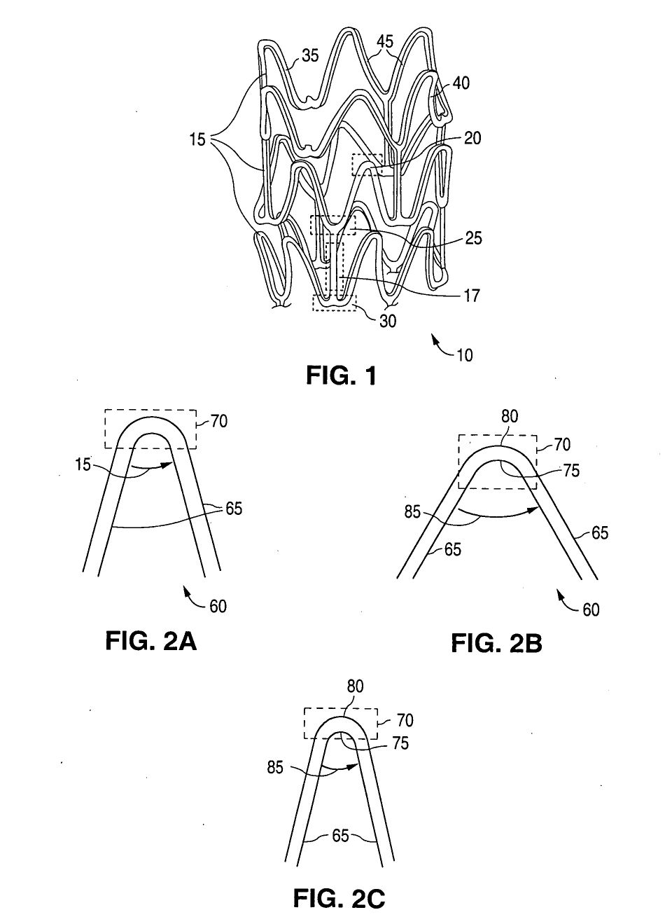

A stent may have a pattern that includes a number of interconnecting elements. Variations of the structure of such patterns are virtually unlimited. FIG. 1 depicts a three-dimensional view of a stent 10 to illustrate a conventional stent with a typical stent pattern having interconnecting elements or struts 15. As shown in FIG. 1 the geometry or shape of stents can vary throughout its structure. A pattern may include portions of struts that are straight or relatively straight, an example being a section 17. In addition, patterns may include struts that include curved or bent portions as in a section 20. Patterns may also include intersections of struts with curved or bent portions as in sections 25 and 30. As shown in FIG. 1, struts 15 of stent 10 include luminal faces 35, abluminal faces 40, and side-wall faces 45.

In some embodiments, a pattern of a stent such as that pictured in FIG. 1, may be formed from a tube by laser cutting the pattern of struts in the tube. The stent may also be formed by laser cutting a polymeric sheet, rolling the pattern into the shape of the cylindrical stent, and providing a longitudinal weld to form the stent. Other methods of forming stents are well known and include chemically etching a polymeric sheet and rolling and then welding it to form the stent. The stent may be formed by injection molding of a thermoplastic or reaction injection molding of a thermoset polymeric material.

As indicated above, crimping and expansion of a stent with a typical pattern, such as in FIG. 1, result in localized regions of the pattern having high deformation or strain that are susceptible to failure. The stent pattern depicted in FIG. 1 may be used to illustrate such localized regions. When a stent is crimped or expanded, some portions of a stent pattern may have no or relatively no strain, while others may have relatively high strain. Straight or substantially straight sections of struts such as section 17 of stent 10 in FIG. 1 experience no or relatively no strain. However, sections 20, 25, and 30 may experience relatively high strain when the stent is expanded or crimped.

For example, FIG. 2A depicts a partial planar side view of a luminal or abluminal surface of a portion 60 from a stent in an unexpanded state that includes straight sections 65 and a curved section 70 with an angle 85. When a stent undergoes radial expansion, portions of struts bend resulting in an increase of an angle 85 between straight sections 65, as shown in FIG. 2B. FIGS. 2B-C depict portion 60 in a plane of bending. Radial expansion of a stent causes substantially no strain in straight sections 65. However, the bending of portion 60 causes relatively high stress and strain in most of curved section 70. A concave portion 75 of curved section 70 experiences relatively high tensile stress and strain and a convex portion 80 of curved section 70 experiences relatively high compressive strain. As shown in FIG. 2C, when a stent is crimped, angle 85 decreases and concave portion 75 experiences relatively high compressive strain and convex portion 80 experiences relatively high tensile strain. The boundary between portions 75 and 80 is a surface of zero strain called the neutral axis.

Various embodiments of a method described herein of treating a bodily lumen with a polymeric implantable medical device have several advantages. The embodiments allow treatment with a polymeric device having adequate flexibility during delivery, high radial strength and rigidity (high modulus) after deployment, high creep resistance after deployment. The devices may also be free or relatively free of localized regions of high deformation that are susceptible to failure. Furthermore, the deployed device may have a T_{g} higher than the body temperature which helps maintain the strength and rigidity of the device. Conversely, during delivery the device may be configured to have a T_{g} below body temperature which may allow the device to remain flexible.

Certain embodiments of a method of treating a bodily lumen with an implantable medical device may include disposing the device within a bodily lumen. The device may be delivered within the bodily lumen using a delivery implement such as a catheter balloon. A delivery location within the lumen may be the site of lesion requiring treatment. The device may first be disposed over the catheter balloon prior to delivery.

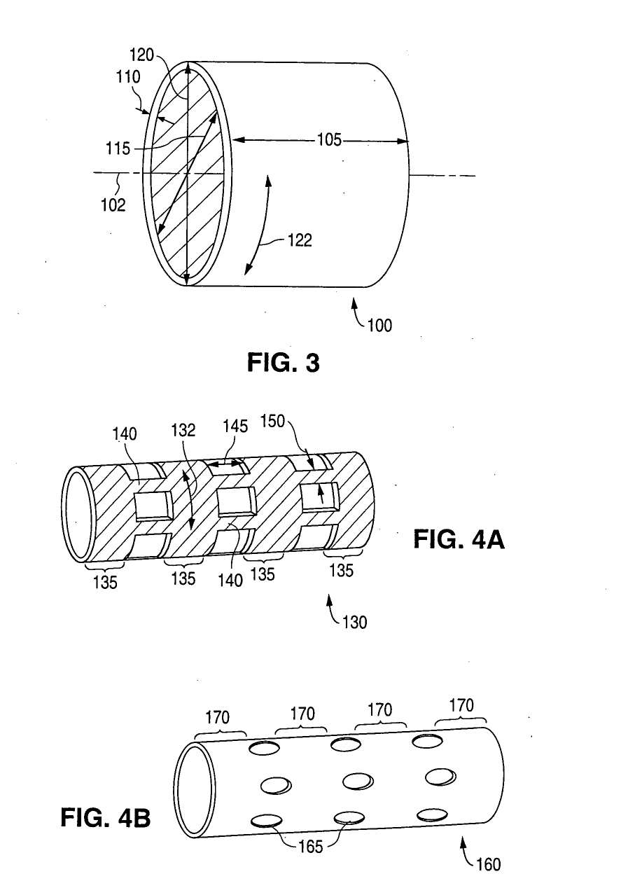

In an embodiment, the device may include a tube-like section having an abluminal face and a luminal face extending between a proximal end and a distal end of the section. A tube-like section may refer to a cylindrical or substantially cylindrical element free or relatively free of holes or voids in a wall of the element. FIG. 3 depicts a tube-like section 100 having a cylindrical axis 102, a length 105, a wall thickness 110, an inside diameter 1115, and an outside diameter 120. Numerous embodiments of a device having a tube-like section are possible.

In an embodiment, the device may include a second tube-like section coupled to the tube-like section. The second tube-like section may be coupled to the tube-like section by at least two connecting elements. In further embodiments, a device may include a plurality of tube-like sections. The tube-like sections may be coupled by connecting elements. FIG. 4A depicts stent 130 having a plurality of tube-like sections 135 joined by connecting elements 140. Connecting elements 140 have a length 145 and a width 150. The length of the tube-like sections and the length and width of the connecting elements can be varied to obtain desired mechanical behavior. For instance, increasing the length of tube-like sections and decreasing the length of connecting elements may increase the radial strength of the stent while reducing the flexibility of the stent.

Additionally, the number of connecting elements around the circumference of the stent may be varied as well. Also, the dimensions of the tube-like sections and connecting elements may vary along the axis of the stent. In this way different mechanical requirements relating to treatment of a lesion, for example, along the axis of the stent may be accommodated.

FIG. 4B depicts another embodiment of a stent with tube-like sections. Stent 160 has disc-shaped or circular holes 165 separating tube-like sections 170. In one embodiment, a stent may be made up substantantially or completely of a single tube-like section.

Furthermore, the method may further include radially expanding the device within the lumen. In one embodiment, the device may be expanded by circumferentially deforming the tube-like section. The deforming section may expand the lumen. In addition, the deformed section may also support the lumen.

Since the tube-like section has a cylindrical shape, it may be expanded substantially uniformly around its circumference. Thus, the expanded device may be free or relatively free of localized regions of high deformation, as in conventional stents described above.

In an embodiment, an inside diameter of the device including a tube-like section prior to expansion, may be equal or approximately equal to an outside diameter of the delivery implement. Unlike conventional stents described above, it is not necessary to fabricate a device with an inside diameter greater than an outside diameter of a delivery implement such as the balloon catheter. A diameter of a device is increased substantially or completely through circumferential deformation of a tube-like section rather than through changes in angles between structural elements, as illustrated in FIGS. 2A-C.

Furthermore, a device with a tube-like section delivered and deployed as described herein may be free or relatively free of localized regions of high deformation and strain (e.g., sections 20, 25 and 30 in FIG. 1). The localized regions of high deformation and strain are due to structural elements moving with respect to one another during crimpling and deployment. As noted above, such regions are susceptible to failure.

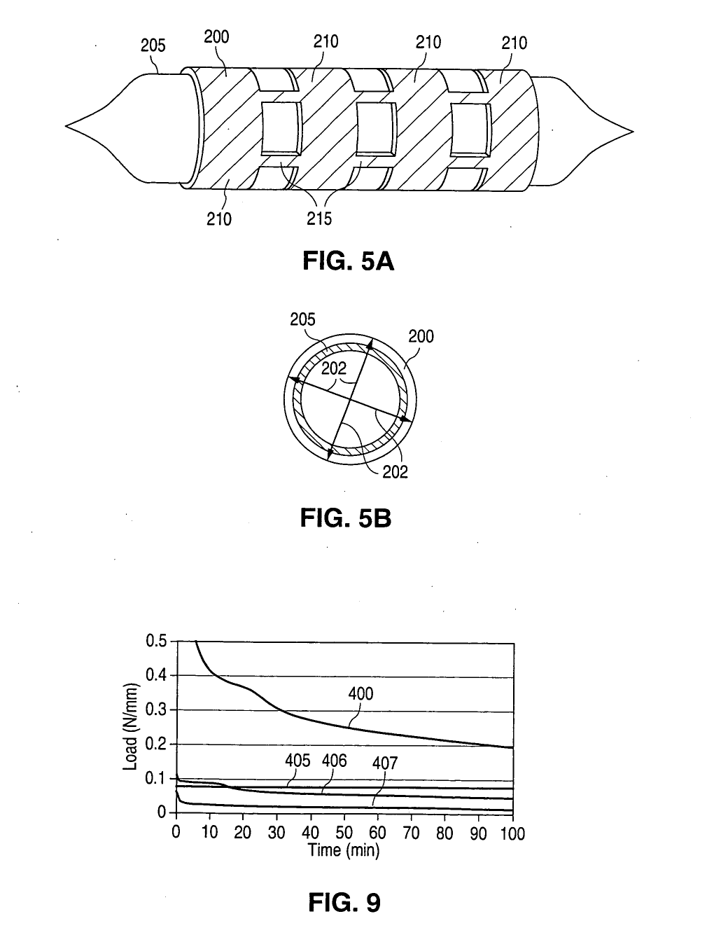

As an illustration, FIG. 5A depicts a stent 200 having tube-like sections 210 and connecting elements 215 disposed over a balloon 205. The radial cross-section in FIG. 5B shows arrows 203 depicting the radial expansion of a tube-like section of stent 200. FIG. 5B also shows an inside diameter of stent 200 is approximately the same as an outside diameter of balloon 205.

As indicated above, polymers tend to be flexible above their T_{g}. As indicated above, increasing a temperature of a polymer may facilitate expansion and inducing orientation and crystallization. Therefore, it is desirable that the polymer of the device be above its T_{g} during delivery of the stent. In some embodiments, the polymer of the device may have a T_{g} at or below body temperature.

In some embodiments, the device may be fabricated from a polymer blend. The T_{g} of the blend may be tuned by adjusting the relative weight percent of the components in the blend. The T_{g} of a blend may be predicted using a suitable equation, for example, the Fox equation:
1/T_{g}=Wₐ/T_{ga}+W_{b}/T_{gb}
where T_{ga} and T_{gb} are the glass transition temperatures of polymers “a” and “b”, and Wₐ and W_{b} are the weight fraction of polymers “a” and “b.”

In certain embodiments, the device may be heated through contact with the body upon insertion into the body. Some embodiments may include heating the device prior to delivering the device into a body. Other embodiments may include heating the device during delivery and/or during expansion of the device. The device may be heated by pumping a heated fluid into the delivery implement.

In some embodiments, the radial expansion of a tube-like section may induce crystallization and/or circumferential molecular orientation around a circumference of the tube-like section. For example an arrow 122 in FIG. 3 or arrow 132 is FIG. 4A depicts the direction of induced orientation of polymer chains in a tube-like section. As discussed above, induced crystallization and orientation tend to increase the mechanical strength and rigidity of tube-like section along the direction of orientation of the polymer chains. Therefore, the radial strength and rigidity of a tube-like section may be increased by expansion of the device.

The induced orientation and crystallization may further favorably modify the device. After expansion in a lumen, it is generally desirable for a stent to be remain rigid and maintain its expanded shape so that it may continue to hold open the lumen. Induced orientation and crystallization of a tube-like section may increase a T_{g} of at least a portion of a tube-like section.

In some embodiments, the T_{g} of the polymer in the device may be increased to above body temperature. Therefore, barriers to polymer chain mobility below T_{g} inhibit or prevent loss of induced orientation and crystallization. Thus, an expanded tube-like section may have a high creep resistance and may more effectively resist radial compressive forces and retain the expanded shape during a desired time period.

In addition, in some embodiments, the device and tube-like sections may be expanded plastically, allowing the device to retain its expanded shape. In other embodiments, the device may be elastically expanded. However, as pointed out above, even an elastically expanded device may retain its expanded shape if its temperature is below its T_{g}. Thus, applying strain-induced crystallization to a polymer sample may result in a permanently deformed high strength, high modulus material with a higher T_{g} than the original sample.

Additionally, it was pointed out above that it is desirable for a stent to have sufficient flexibility during delivery. Embodiments of the method described above allow a stent be flexible during delivery as well as be strong and rigid after deployment. The expansion that induces strength and rigidity is performed only after a device is delivered to a desired location where it is expanded.

In one embodiment, the flexibility of the device may be increased by modifying parameters of a pattern of tube-like sections and/or connecting elements, as discussed above. For example, decreasing a length 105 of tube-like sections as shown in FIG. 3 may increase the flexibility of a device depicted in FIG. 4. In other embodiments, a stent with desired flexibility may be obtained by using a polymer for the device with a T_{g} below a selected temperature. As indicated above, polymers tend to be more flexible below a T_{g} of the polymer. Therefore, a selected temperature may be less than or equal to an ambient temperature. Alternatively, the selected temperature may be between an ambient temperature and a body temperature.

In certain embodiments, a tube-like section may be composed of more than one layer. One embodiment may include a tube-like section having an abluminal layer and a luminal layer. In another embodiment, a tube-like section may include at least one inner layer between the abluminal layer and the luminal layer.

In one embodiment, a stent having more than one layer may be fabricated from a multilayer tube. The multilayer tube may be formed by co-extruding different polymers corresponding to each layer into a single polymer tube.

One embodiment may include alternating stiff and strong layers having relatively high moduli with flexible layers having relatively low moduli. Since a stiff and strong layer may be susceptible to fracture, it may be desirable for an outermost layer to be a flexible layer.

In certain embodiments, the abluminal, the luminal, and inner layers may be formed from bioabsorbable polymers. One embodiment may include an inner layer with a different average erosion rate or a different half-life than the abluminal and/or the luminal layer. In some embodiments, the abluminal and luminal layers may be configured to delay, inhibit, or prevent erosion of the inner layer in a manner that allows the inner layer to provide mechanical support to a bodily lumen for a desired time period.

### EXAMPLES

Some embodiments of the present invention are illustrated by the following Example. The Example is being given by way of illustration only and not by way of limitation. The parameters and data are not to be construed to unduly limit the scope of the embodiments of the invention.

### Example 1

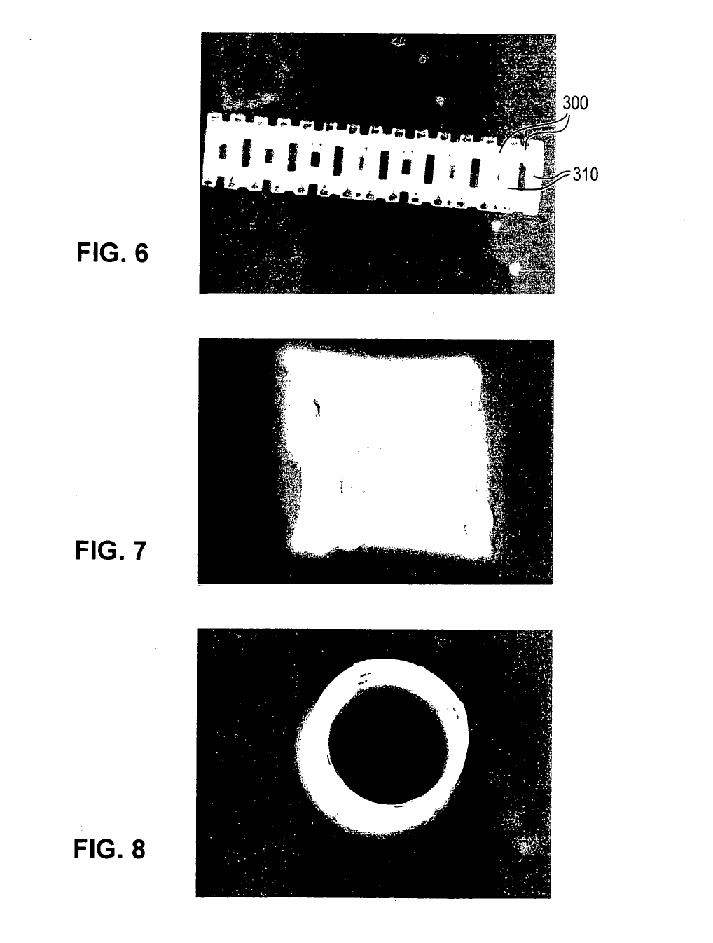

A tube with an outside diameter (OD) of 0.064 inch and an inside diameter 0.038 inch (ID) was manufactured from a blend of two polymers (80 wt % poly(L-lactide) and 20 wt % poly(L-lactide-co-trimethylene carbonate). The stent was laser cut from the tube. FIG. 6 depicts a scanning optical microscope image of a stent after laser cutting the tube. The stent has a plurality of tube-like sections 300 connected by connecting elements 310.

The stent was mounted on a 3.0×18 mm balloon which was inflated to 20 atmospheres at 50° C. which was above the T_{g} of the polymer. The balloon was heated by immersing it in a water bath. FIGS. 7 and 8 depict scanning optical microscope images of the stent after expansion. FIG. 7 depicts a side view and FIG. 8 depicts a view down the cylindrical axis of the expanded stent.

Expansion of the stent increased the diameter of the stent. FIG. 7 shows that expansion caused a decrease in length of the stent and “dogboning,” i.e., the increase in the diameter appears to be slightly larger at the ends of the stent. The temporary dogbone shape results when the ends of the stent expand before the center. The dogboning is due to an imperfect match between the length of the stent and the length of the balloon. The dogboning may be substantially eliminated by matching the stent length with the balloon length.

The influence of the radial expansion of the stent on long term creep was investigated. Long term creep tests were performed with an Instrom testing machine obtained from Instron in Canton, Mass. that was controlled with Merlin™ Materials Testing Software. The expanded stent was placed between two flat plates. The plates were adjusted to compress the stent by 20%, i.e., equivalent to a 20% reduction in diameter. A resistance force, the amount force in units of Newtons/mm that was necessary to keep the stent at a 20% compression, was measured as a function of time. Tests were also performed on a metallic stent and two prototype polymeric stents.

FIG. 9 depicts the results of the long term creep tests for the expanded stent (curve 400), the metallic stent (curve 405), prototype polymeric stent-1 (curve 406), and prototype polymeric stent-2—(curve 407). The properties of the stents are shown in Table 1. FIG. 9 shows that the expanded stent is substantially stronger than the metallic stent and the prototype polymeric stents. Consequently, the expanded stent would have a higher resistance to radially inward compressive forces of vessel walls. Additionally, the initial decrease in the resistance force or creep for the prototype polymeric stents (curves 406 and 407) is relatively more substantial than for the expanded stent (curve 400). Therefore, the expanded stent has a higher radial strength than the metallic and prototype polymeric stents and a higher creep resistance than the prototype polymeric stents.

TABLE 1
Stents used in long term creep tests.

| Stent | Material |
|---|---|
| Expanded Stent | Poly(L-lactide) |
| Metallic stent: SS-Multi 3.0 | Stainless Steel |
| (Stainless Steel Multi-link) | |
| Prototype Polymeric Stent (1) | 100% Poly(L-lactide) HG F-71 |
| Prototype Polymeric Stent (2) | 100% Poly(L-lactide) HG F-71 |
| | E-Beam 3.0 |

Additionally, differential scanning calorimetry (DSC) was used to study the increase in the T_{g} due the radial orientation induced by the radial expansion. In general, DSC is a technique that may be used to identify thermal transitions in a polymer. Thermal transitions include, for example, crystallization, and melting. A thermal transition in a polymer may be endothermic (sample absorbs heat) or exothermic (sample expels heat). Glass and melting transitions are exothermic and crystallization is endothermic.

In a typical DSC run, a polymer sample is heated at a constant rate. The heat inflow or outflow into the sample is controlled to keep the heating rate constant. When the sample undergoes a thermal transition, heat is either absorbed or expelled. At the glass transition and melting transition, heat flow into the sample decreases. When a polymer sample crystallizes, the heat flow into the sample increases.

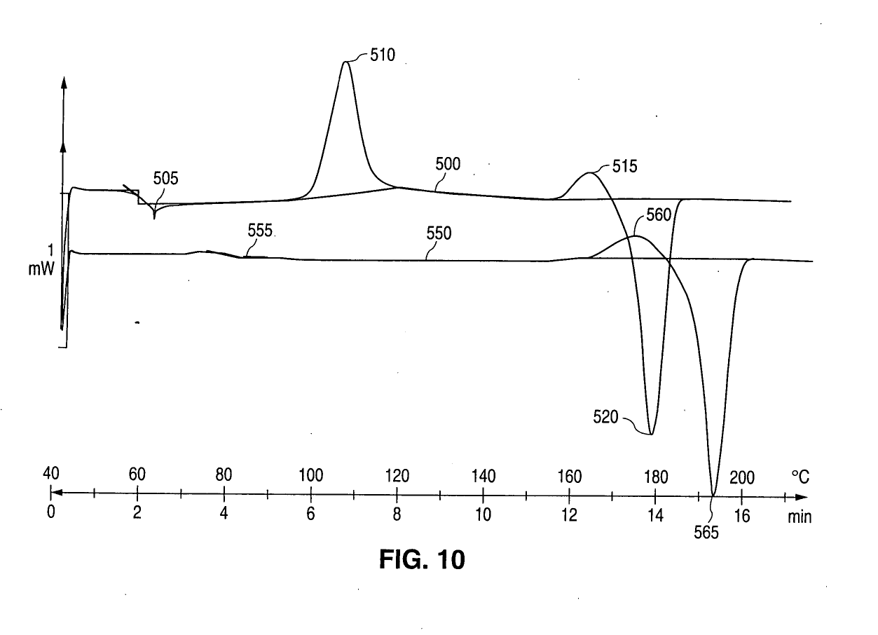

FIG. 10 depicts the results of DSC runs for samples including the stent shown in FIG. 6 before expansion (curve 500) and the stent after expansion (curve 550). The vertical axis is the heat energy flow in milliwatts into or out of a sample. The horizontal axis includes both the time a sample has been heated and an instantaneous temperature of a sample. A trough 505 in curve 500 corresponds to the glass transition in the stent that has not been expanded. The onset of the transition is at 56.77° C. and the midpoint of trough 505 is at 58.66° C. A trough 555 in curve 550 corresponds to the glass transition in the expanded stent. The onset of the transition is at 61.96° C. and the midpoint of trough 555 is at 63.25° C. Radial expansion of the stent has increased the glass transition temperature by about 4.6° C.

In addition, curve 500 has a peak 510 which corresponds to the crystallization transition of the stent. Curve 550 does not have an analogous peak since crystallization was induced by radial expansion.

Peak 515 in curve 500 corresponds to an additional crystallization transition. The oriented polymer has a similar transition as shown by a peak 560 in curve 550. Even after initial crystallization, imperfections may be present in the crystal lattice. The transition at peak 560 corresponds to further reorganization of the crystalline structure that tends to occur at higher temperatures. Trough 520 in curve 500 and trough 565 in curve 550 correspond to melting transitions for the respective samples.

### Example 2

An alternative material of construction for a stent is a copolymer of poly (L-lactide) and poly(ε-caprolactone). The T_{g} of this material may be tuned to a desired value using the Fox equation discussed above or some other suitable equation.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method of treating a bodily lumen with an implantable medical device comprising:
disposing an implantable medical device within a bodily lumen, the device comprising a tube-like section having an abluminal face and a luminal face extending between a proximal end and a distal end of the section; and
radially expanding the device about a cylindrical axis of the section within the lumen by circumferentially deforming the section, wherein the deforming section expands the lumen and the deformed section supports the lumen.

2. The method of claim 1, wherein the device is a stent.

3. The method of claim 1, wherein the device comprises a second tube-like section coupled to the tube-like section by at least two connecting elements.

4. The method of claim 1, further comprising disposing the implantable medical device over a delivery implement before disposing the device within the lumen.

5. The method of claim 4, wherein the delivery implement comprises a balloon catheter.

6. The method of claim 1, wherein the section comprises a biostable polymer and/or a bioabsorbable polymer.

7. The method of claim 1, wherein the radial expansion induces crystallization and/or circumferential molecular orientation in the section.

8. The method of claim 1, wherein the deformation increases a circumferential mechanical strength and/or modulus of the section.

9. The method of claim 1, wherein the material comprises a polymer, and wherein the deformation increases a T_{g} of at least a portion of the section.

10. The method of claim 1, wherein the deformation of the section comprises plastic deformation.

11. The method of claim 1, wherein the section comprises an abluminal layer, a luminal layer, and optionally, at least one inner layer, wherein the abluminal layer comprises a different polymer than the luminal layer, and wherein at least one inner layer comprises a different polymer than the abluminal layer and/or luminal layer.

12. A method of fabricating a system for treating a bodily lumen comprising:
disposing an implantable medical device over a delivery implement, the device comprising a tube-like section having an abluminal face and a luminal face extending between a proximal end and a distal end, the delivery implement configured to radially expand the device within the lumen by circumferentially deforming the section about a cylindrical axis of the section, wherein the deforming section expands the lumen and the deformed section supports the lumen.

13. The method of claim 12, wherein the device is a stent.

14. The method of claim 12, wherein the delivery implement comprises a balloon catheter.

15. The method of claim 12, wherein an inside diameter of the section before expansion is equal or approximately equal to an outside diameter of the delivery implement.

16. The method of claim 12, wherein the device comprises a second tube-like section coupled to the tube-like section by at least two connecting elements.

17. The method of claim 12, wherein the section comprises a biostable polymer and/or bioabsorbable polymer.

18. A system for treating a bodily lumen comprising:
an implantable medical device having a tube-like section, the device comprising a tube-like section having an abluminal face and a luminal face extending between a proximal end and a distal end of the section;
a delivery implement configured to radially expand the device by circumferentially deforming the section about a cylindrical axis the section, wherein the deforming section expands the lumen and the deformed section supports the lumen.
